(19) United States
(12) Patent Application Publication
(10) Pub. No.: US 2025/0146982 A1
(43) Pub. Date: May 8, 2025

(54) AUTOMATED ONLINE MEASUREMENT OF RESIDUAL CORROSION INHIBITORS IN AQUEOUS OIL FIELD FLUIDS

(71) Applicant: Saudi Arabian Oil Company, Dhahran (SA)

(72) Inventors: Joseph Joseph Puthuvelil, Udhailiyah (SA); Kishore B. Gudapati, Al Hufuf (SA); Abdulaziz A. Alsalman, Al Mubarraz (SA)

(21) Appl. No.: 18/502,884

(22) Filed: Nov. 6, 2023

(51) Int. Cl. G01N 30/16; B01D 15/16; B01D 15/32; G01N 30/02; G01N 30/32; G01N 30/74; G01N 33/28

(52) U.S. Cl. CPC G01N 30/16; B01D 15/166; B01D 15/325; G01N 30/32; G01N 30/74; G01N 33/2823; G01N 2030/027; G01N 2030/326

(57) ABSTRACT

Systems for determining the concentration of a corrosion inhibitor can include an autosampler and an liquid chromatography (LC) system. The autosampler can sample an aqueous liquid having a corrosion inhibitor from a fluid stream of an oil field system into an injector of a LC system. An injector injects aqueous liquid of a predetermined volume and a mobile phase into an LC system column. Over a first predetermined time, the first solvent concentration range is increased to a second solvent concentration range and the first buffer concentration range is decreased to a second buffer concentration range. A detector determines a concentration of corrosion inhibitor in the aqueous liquid flowing out of the column. Over a second predetermined time, the second solvent concentration range is decreased to the first solvent concentration range over a second predetermined time, and the second buffer concentration range is increased to the first buffer concentration range.

### BACKGROUND

### Technical Field

The present disclosure applies to systems and techniques for characterizing a quantity of corrosion inhibitor in aqueous-based oil field fluids.

### Background

Corrosion inhibiting chemicals are used to protect metal surfaces that come into contact with oil field fluids in oil and gas production systems. Nitrogen containing components are commonly used in Corrosion inhibitor formulations which include amides, imidazoline, salts of nitrogenous molecules, nitrogen quaternaries, polyoxyalkylated amines, amides and imidazolines, nitrogen heterocyclic, etc.

High-performance liquid chromatography (HPLC), formerly referred to as high-pressure liquid chromatography, is a technique in analytical chemistry used to separate, identify, and quantify each component in a mixture. HPCL relies on pumps to pass a pressurized liquid solvent (mobile) containing the sample mixture through a column filled with a solid adsorbent material (stationary phase). Each component in the sample mixture interacts slightly differently with the adsorbent material, causing different flow rates for the different components and leading to the separation of the components as they flow out of the column. A detector generates a signal proportional to the amount of sample component emerging from the column, hence allowing for quantitative analysis of the sample components. A digital microprocessor and user software control the HPLC instrument and provide data analysis. Some models of mechanical pumps in an HPLC instrument can mix multiple solvents together in ratios changing in time, generating a composition gradient in the mobile phase. Various detectors are in common use, such as UV/Vis, photodiode array (PDA) or based on mass spectrometry.

In HPLC, the stationary phase includes more polar materials, while the mobile phase is non-polar. In reverse-phase liquid chromatography (RPLC), the stationary phase is (more) non-polar, while the mobile phase is (more) polar.

### SUMMARY

The present disclosure describes techniques that can be used for selective detection of corrosion inhibitor chemicals within an aqueous-based flow stream of an oil field system.

In some implementations, a computer-implemented method includes the following:

Aspects of the implementations are directed to method for determining the concentration of a corrosion inhibitor, the method including receiving an aqueous liquid of a predetermined volume from a fluid stream of an oil field system into an injector of a liquid chromatography system, the aqueous liquid includes a corrosion inhibitor, the liquid chromatography system in fluid communication with the fluid stream of the oil field system, the liquid chromatography system includes a stationary phase and a mobile phase, the mobile phase includes a solvent at a first solvent concentration range and a buffer at first buffer concentration range; injecting, by the injector, the aqueous liquid of the predetermined volume and the mobile phase into a column of the liquid chromatography system; increasing, by a controller controlling a pump system, the first solvent concentration range to a second solvent concentration range over a first predetermined amount of time, and decreasing, by the controller controlling a pump system, the first buffer concentration range to a second buffer concentration range over the first predetermined amount of time; determining, by a detector of the liquid chromatography system, a concentration of corrosion inhibitor in the aqueous liquid flowing out of the column; and decreasing, by the controller controlling the pump system, the second solvent concentration range to the first solvent concentration range over a second predetermined amount of time, and increasing, by the controller controlling the pump system, the second buffer concentration range to the first buffer concentration range over the second predetermined amount of time.

A detection system for determining a concentration of corrosion inhibitor in an aqueous liquid from a fluid steam of an oil field system, the detection system including a liquid chromatography (LC) subsystem including an LC column includes a non-polar stationary phase, an injector to inject a mobile phase and aqueous liquid into the LC column, a controller to control a time rate of change of a relative concentration of solvent and buffer solution in the mobile phase, a detector to detect a concentration of corrosion inhibitor in the aqueous liquid flowing from the column during operation of the LC subsystem, and an autosampler to receive aqueous liquid from a fluid stream of the oil field system and to deliver the aqueous liquid to the liquid chromatography subsystem. The detection system also includes a fluid inlet coupler to couple an inlet of the autosampler to an outlet of an asset of the oil field system, the asset containing the fluid stream.

In some implementations, the first solvent concentration range includes a concentration of solvent in the mobile phase ranging from 24% to 26% and the first buffer concentration range includes a concentration of buffer solution in the mobile phase ranging from 74% to 76%.

In some implementations, the second solvent concentration range includes a concentration of solvent in the mobile phase ranging from 84% to 86% and the second buffer concentration range includes a concentration of buffer solution in the mobile phase ranging from 14% to 16%.

In some implementations, the first predetermined amount of time is between 24 and 26 minutes.

In some implementations, the injector injects the aqueous liquid of the predetermined volume and the mobile phase into a column of the liquid chromatography system at a flow rate of approximately 0.8 milliliters per minute (mL/min).

In some implementations, the predetermined volume includes approximately 1 microliter of aqueous liquid.

In some implementations, the detector operates at 280 nanometers (nm) wavelength and a reference wavelength of 600 nanometers.

In some implementations, the autosampler controls a metering pump to sample the predetermined volume of the aqueous liquid from the fluid stream.

In some implementations, the metering pump is coupled to a sample reservoir in fluid communication with the fluid stream, the sample reservoir to receive aqueous liquid from the fluid stream through a reservoir inlet and return aqueous fluid to the fluid stream through a reservoir return outlet.

In some implementations, the fluid stream of the oil field system includes one or more of a fluid stream flowing out of water-oil-separation tank, a fluid stream flowing out of a water degassing tank, a fluid flowing through a crude oil inlet to a gas-oil separation plant (GOSP), or a fluid flowing through a crude oil transfer pipeline.

In some implementations, the second predetermined amount of time is between 4 and 6 minutes.

In some implementations, the solvent includes acetonitrile.

In some implementations, the buffer includes ammonium acetate.

In some implementations, the corrosion inhibitor includes one or more of amides, imidazoline, salts of nitrogenous molecules, nitrogen quaternaries, polyoxyalkylated amines, amides and imidazolines, nitrogen heterocyclic.

In some implementations, the stationary phase is non-polar.

Some implementations also include a metering pump downstream of the autosampler to control the volume of aqueous liquid delivered to the liquid chromatography subsystem.

Some implementations also include a pump system controlled by the controller to pump the solvent and the buffer into the injector, the controller to control the pump system to increase a relative concentration of the solvent from 25% to 85% over 25 minutes and to decrease a relative concentration of the buffer solution from 75% to 15% over the 25 minutes.

In some implementations, the liquid chromatography subsystem includes a reverse phase liquid chromatography (RPLC) subsystem.

The previously described implementation is implementable using a computer-implemented method; a non-transitory, computer-readable medium storing computer-readable instructions to perform the computer-implemented method; and a computer-implemented system including a computer memory interoperably coupled with a hardware processor configured to perform the computer-implemented method/the instructions stored on the non-transitory, computer-readable medium.

The subject matter described in this specification can be implemented in particular implementations, so as to realize one or more of the following advantages. For example, the techniques described herein provide for a portable, on-site corrosion detection system that can monitor the concentration of corrosion inhibitor chemicals within an aqueous-based flow stream flowing through various locations of an oil field system. In addition, the techniques described herein can monitor and detect corrosion inhibitor chemicals at low concentrations without using a reagent. Such detection can reduce health, safety, and environmental risks associated in shipping samples of aqueous liquids to centralized testing laboratories, while also saving on time-to-detection. The utilization of online methods can yield immediate inhibitor residual results, leading to enhanced system control and improved corrosion management inside the facility.

The details of one or more implementations of the subject matter of this specification are set forth in the Detailed Description, the accompanying drawings, and the claims. Other features, aspects, and advantages of the subject matter will become apparent from the Detailed Description, the claims, and the accompanying drawings.

### DESCRIPTION OF DRAWINGS

FIG. 1 is a schematic diagram of an example corrosion inhibitor detection system, according to some implementations of the present disclosure.

FIG. 2 is a schematic diagram of an example oil field system that illustrates example fluid stream locations for analyzing aqueous liquid for corrosion inhibitor concentration in accordance with some implementations of the present disclosure.

FIG. 3 is a graphical representation of an example calibration curve for establishing a relative concentration of the mobile phase in accordance with some implementations of the present disclosure.

FIG. 4 is an example chromatogram showing retention time of corrosion inhibitors samples using the corrosion inhibitor detector in accordance with some implementations of the present disclosure.

FIG. 5 is a process flow diagram for detecting a concentration of corrosion inhibitor in an aqueous liquid from a fluid stream of an oil field system in accordance with some implementations of the present disclosure.

FIG. 6 is a block diagram illustrating an example computer system used to provide computational functionalities associated with described algorithms, methods, functions, processes, flows, and procedures as described in the present disclosure, according to some implementations of the present disclosure.

Like reference numbers and designations in the various drawings indicate like elements.

### DETAILED DESCRIPTION

The following detailed description describes techniques for characterizing a quantity of corrosion inhibitor in aqueous-based oil field fluids. Various modifications, alterations, and permutations of the disclosed implementations can be made and will be readily apparent to those of ordinary skill in the art, and the general principles defined may be applied to other implementations and applications, without departing from scope of the disclosure. In some instances, details unnecessary to obtain an understanding of the described subject matter may be omitted so as to not obscure one or more described implementations with unnecessary detail and inasmuch as such details are within the skill of one of ordinary skill in the art. The present disclosure is not intended to be limited to the described or illustrated implementations, but to be accorded the widest scope consistent with the described principles and features.

Corrosion inhibiting chemicals are used to protect metal surfaces that come into contact with oil field fluids in oil and gas production systems. A strong corrosion management program includes four key components: 1) corrosion residual measurements downstream of injection, 2) tank level monitoring, 3) corrosion rate monitoring using techniques such as electrical resistance (ER) probes, linear polarization resistance (LPR) probes, and weight loss coupons, and 4) microbial growth monitoring. While tank level monitoring, corrosion rate monitoring, and microbiological monitoring are generally well established, measuring corrosion inhibitor residuals in ppm levels from oilfield crudes and complicated brines remains difficult. However, the residual concentration of corrosion inhibitors is useful in monitoring performance efficiency and the relevance of keeping a minimum inhibitor concertation of the chemical in the fluid to safeguard the pipeline and equipment system.

Nitrogen containing components are commonly used in Corrosion inhibitor formulations which include amides, imidazoline, salts of nitrogenous molecules, nitrogen quaternaries, polyoxyalkylated amines, amides and imidazolines, nitrogen heterocyclic, etc. In recent years, several developments in the identification and monitoring of residual corrosion inhibitors in oilfield fluids have been documented. The main techniques such as Gas chromatography and mass spectrometry techniques (GC-MS) high performance Liquid Chromatography (HPLC) and Liquid chromatography mass spectrometry. Although most of these methods offer high resolution molecular detail with great sensitivity, however, they lack portability, and are off site, time consuming laboratory procedure.

The developed automated method is specific for detection and monitoring of nitrogen containing corrosion inhibitors (quaternary amines, imidazolines, amides etc.) in aqueous based oil filed fluids. The method employed is based on the retention of the nitrogen-based corrosion inhibitor species in the stationary phase of the LC column. The disclosure describes the direct and continuous measurement of the concentration of inhibitor in aqueous fluids. Herein is described the procedure and equipment for accurately detecting nitrogen based corrosion inhibitor as well as field residual data which provided information leading to process adjustments and improvements.

This innovative automated online method is capable of detecting, quantifying and monitoring residual corrosion inhibitor in aqueous based oil field fluids. The developed method can be used for monitoring nitrogen based chemical formulations in a corrosion inhibitor. The system is equipped with an online sampling system connected with a customized LC instrument. An autosampler is provided to prepare a sample of fluid containing corrosion inhibitor and placing the exposed sample into the LC instrument. Corrosion inhibitor concentrations data are obtained from the aqueous liquid sample though a PC system. The concentration of corrosion inhibitor in the fluid is determined using liquid chromatographs, such as reversed phase liquid chromatography. The liquid chromatography can use a solvent and a buffer solution with a concentration gradient. As an example, Acetonitrile solvent and Ammonium acetate buffer solution can be used, with gradient relative concentrations. The relative concertation of the solvents is varied to predetermined gradient, as described below. The techniques described herein do not require any chemical reagents; rather, the techniques use a solvent and buffer solution in predefined relative concentrations.

This technique can use an online measurement within the oil field system at various locations to provide reliable data for the corrosion control. The data can be connected corrosion control compliance index (CCCI) to manage corrosion treatment in oil and gas production and refining systems. The method is suitable to detect the chemical in low concentrations in aqueous oil field fluid ranges from 0.5 ppm to 500 ppm. The method is specific to nitrogen containing components present in corrosion inhibitor, but can be refined to apply to other prevalent species. Some implementations have exhibited no interference from H₂S or High Total Dissolved Solid samples.

The method is suitable to detect the chemical in low concentrations ranges from 0.5 ppm to 500 ppm. The method will reduce the health, safety and environmental risk associated in shipping samples to the centralized laboratories. No interference from H₂S or High TDS (Total Dissolved Solid) samples.

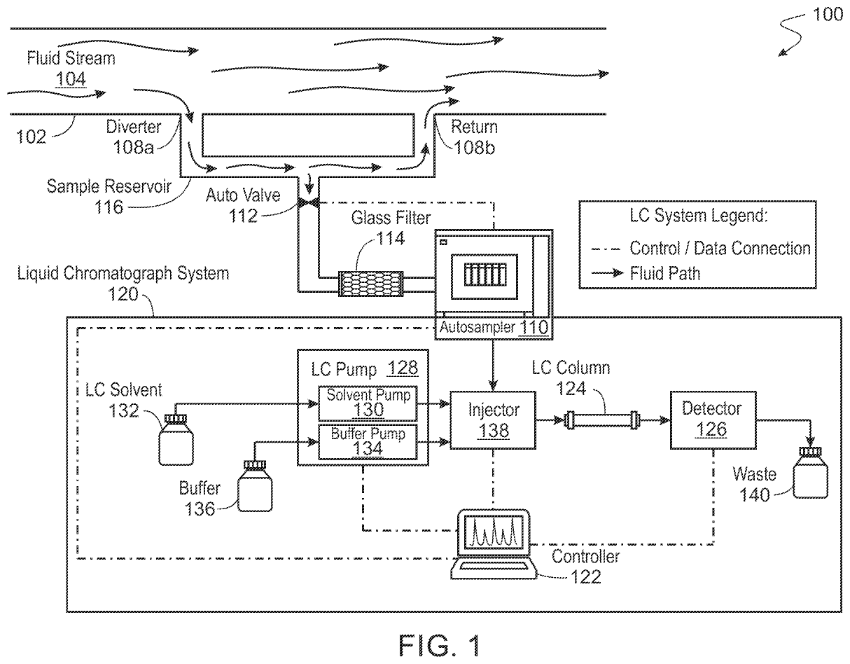

FIG. 1 is a schematic diagram of an example corrosion inhibitor detection system 100, according to some implementations of the present disclosure. The corrosion inhibitor detection system 100 can be removably coupled to a sample point of an oil field system asset 102. The oil field system asset 102 can include a diverter 108 and a return 108b at a sampling point (e.g., connected via a quick connector or other tap point). A sample reservoir 116 can be coupled to the diverter 108. Aqueous liquid can be diverted into the sample reservoir 116 and can be sampled for analysis from the sample reservoir 116. The remaining aqueous liquid can be returned to the fluid stream 104 via a return 108b.

The corrosion inhibitor detection system 100 includes a liquid chromatography (LC) subsystem 120. The LC subsystem 120 can include a high-pressure liquid chromatography (HPLC) system, such as a reverse phase liquid chromatography (RPLC) system. The LC subsystem 120 can include an LC column 124 that includes a stationary phase with spherical silica particles. In some implementations, the spherical silica can be linked to non-polar and/or hydrophobic molecules, such as alkyl chains.

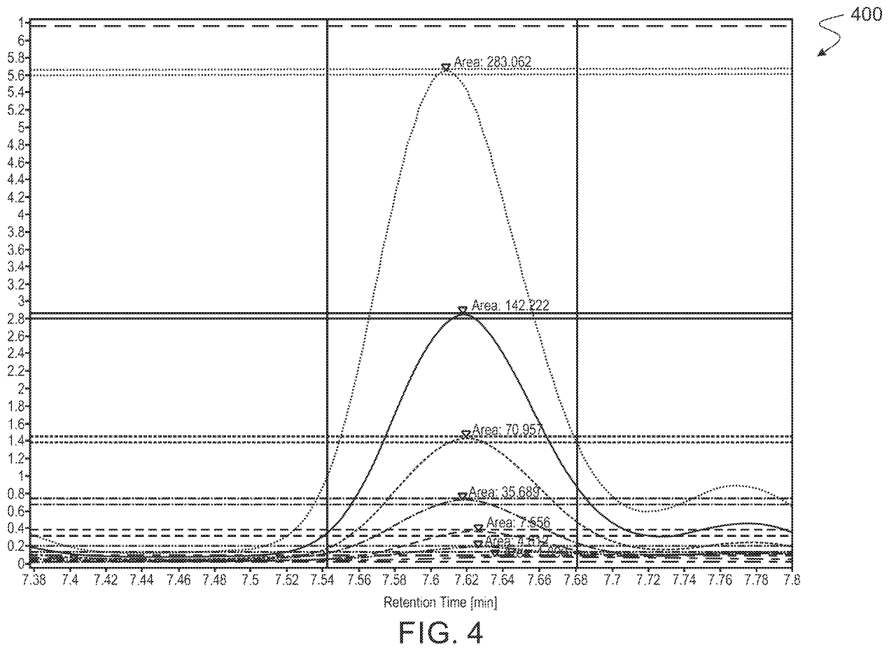

The LC subsystem 120 can include a detector 126 for performing optical detection of analytes output from the LC column 124. The detector 126 can include a non-destructive detector that emits light that is absorbed or scattered by the solution output from the column. The detector can detect the light transmitted through the column, and can infer the absorption of emitted light by the sample. The detector can correlate the amount of light either transmitted or absorbed into an electrical signal representative of the quantity of the analyte in the sample. The spectrogram or chromatogram, shown in FIG. 4, plots the absorption unit against the retention time. Retention time is a quantity of time between injection of the analyte and detection of the analyte. The retention time can be an indicator of what the analyte is. The absorption unit can be an indicator of the quantity or concentration of the analyte.

For example, the detector 126 can emit and detect light through the mobile phase prior to the start of elution to establish a baseline. The detector 126 can then emit and detect light through the sample. A comparison of the results can be made (e.g., by detector 126 or by the controller 112) to determine the quantity or concentration of analyte material in the sample.

The detector 126 can emit light at a wavelength selected for the analyte. In some implementations, the detector 126 can operate at wavelength of 280 nanometers, a reference wavelength of 600 nanometers, and a reference bandwidth 100 nanometers, with a 10 Hz data acquisition sampling rate.

The LC subsystem 120 can include an autosampler 110. The sampling line of the autosampler 110 is connected with the pipe 102 of an oil field system. The sampling line of the autosampler 110 can communicate aqueous liquid from an oil field fluid stream 104 at a 6 o'clock or 6 port position of the autosampler 110 relative to the fluid stream 104. The autosampler 110 can be programmed to automatically deliver a predetermined volume of aqueous liquid at predetermined testing intervals into the LC subsystem 120 for analysis. For example, the autosampler 110 can be programmed to sample one (1) microliter (μL) of aqueous liquid from the fluid stream 104 every thirty minutes.

The autosampler 110 can include or be connected to a metering pump coupled to an auto valve 112. The metering pump can include an inlet in fluid communication with the sample reservoir 116 and an outlet. The outlet of the sample stream is passed through 0.45 μm filtrations system 114 and leading to the LC subsystem 112. LC system suction pump is designed to withdraw 1 micro liter of samples at every 30 minutes intervals.

The LC subsystem 120 can include an LC pump 128. LC pump 128 can include a solvent pump 130 for pumping a solvent 132 into the injector 138 at a predetermined rate. LC pump 128 can include a buffer solution pump 134 for pumping a buffer solution 136 into the injector 138 at a predetermined rate. The solvent 132 and the buffer solution 136 can be mixed for form a mobile phase of the LC subsystem 120. An example of a solvent 132 includes acetonitrile. An example of a buffer solution 134 includes an ammonium acetate solution.

The LC subsystem 120 includes an injector 138. The injector 138 can inject

Examples of the LC subsystem 120 modules include:

The Agilent 1290 Infinity II LC™ used for the experiments consisted of the following modules:
Agilent 1290 Infinity II High-Speed Pump;
Agilent 1290 Infinity II Multi-sampler;
Agilent 1290 Infinity II Multicolumn Thermostat; and
Agilent 1290 Infinity II Diode Array Detector, equipped with a 10-mm Max-Light cartridge cell.

Columns: Agilent Poroshell 120 EC-C18, 4.6×150 mm, 4 μm.

Software: Agilent OpenLAB LC series.

The LC subsystem 120 includes a controller 122. Controller 122 can include a processor and a memory. Controller 122 can be programmed to control one or more modules of the LC subsystem 120. For example, the controller 122 can control the sampling rate and sampling volume of the autosampler 110. The controller 122 can also control the LC pump 128. The controller 122 can control the gradient of the concentration of the solvent and buffer solutions for the mobile phase. Table 1 illustrates an example gradient of the mobile phase.

TABLE 1
Gradient program-Chromatographic conditions for 4.6*150
mm, 5 μm RPLC column.

| Elapsed Time | Gradient |
|---|---|
| 0 minutes | 75% Buffer, 25% Acetonitrile. |
| 25 minutes | 15% Buffer, 85% Acetonitrile |
| 30 minutes | 75% Buffer, 25% Acetonitrile. |

The first solvent concentration range can include a concentration of solvent in the mobile phase ranging from 24% to 26%. Preferably, the first solvent concentration is 25%. The first buffer concentration range can include a concentration of buffer solution in the mobile phase ranging from 74% to 76%. Preferably, the first buffer concentration is 75%.

The second solvent concentration range includes a concentration of solvent in the mobile phase ranging from 84% to 86%, and preferably 85%. The second buffer concentration range includes a concentration of buffer solution in the mobile phase ranging from 14% to 16%, and preferably 15%.

The first predetermined amount of time is between 24 and 26 minutes, and preferably 25 minutes, for transitioning from the first solvent and buffer concentration ranges to the second solvent and buffer concentration ranges. The second predetermined amount of time is between 4 and 6 minutes, and preferably 5 minutes, for transitioning from the second solvent and buffer concentration ranges to the first solvent and buffer concentration ranges.

Table 2 illustrates the mobile phase concentration gradient, as well as other operating parameters during the corrosion inhibitor analysis.

TABLE 2
RP-LC mobile phase concentration gradient

| Parameter | Value |
|---|---|
| Column | Agilent Poroshell 120 EC-C18, 4.6 × 150 mm, 4 μm |
| Mobile phase | Acetonitrile and Buffer |
| Gradient | 0 minutes: 75% Buffer, 25% Acetonitrile. |
| | 25 minutes: 15% Buffer, 85% Acetonitrile. |
| | 30 minutes: 75% Buffer, 25% Acetonitrile |
| Stop time | 30 minutes: 75% Buffer, 25% Acetonitrile |
| Post time | 5 minutes |
| Flow rate | 0.8 mL/min |
| Injection | 99 μL from stock solution, at 25° C., draw speed 200 |
| volume | μL/min, eject speed 400 μL/m, 10 seconds needle |
| | wash |
| Column | 40° C. |
| temperature | |
| Column max | 400 bar |
| pressure | |
| Detection | 280 nm, ref. wavelength 600, reference bandwidth 100, |
| | 10 Hz |

In some implementations, the Autosampler 110 and High Performance Liquid Chromatography (HPLC) equipment 120 can be enclosed within a mobile cage structure, equipped with wheels, facilitating convenient transportation within various locations in the field.

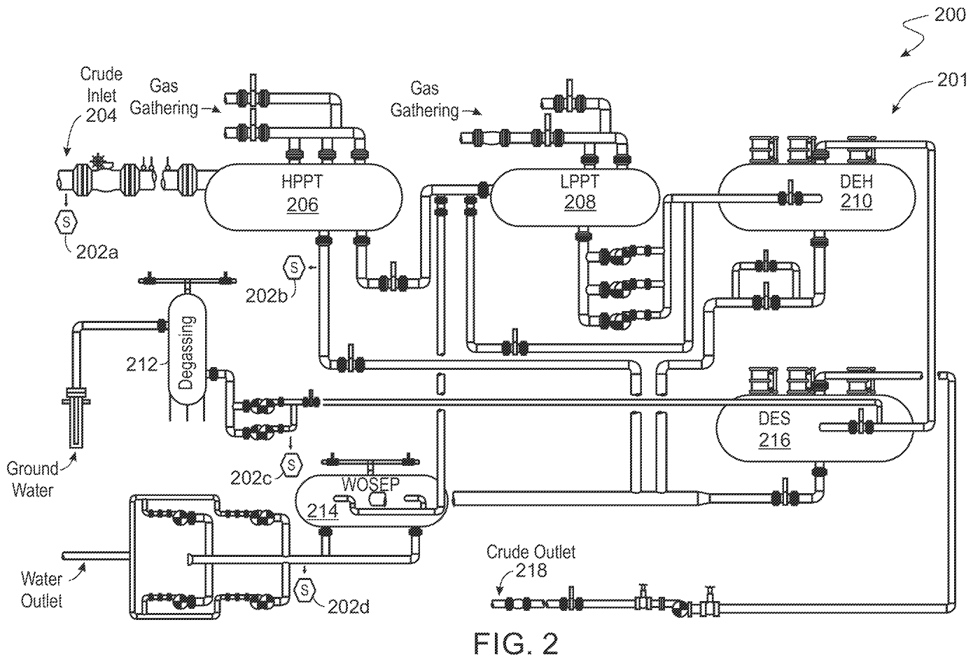

The LC subsystem 100 can be coupled to one or more locations of an oil field system, such as that shown in FIG. 2. FIG. 2 is a schematic diagram of an example oil field system 200 that illustrates example fluid stream locations for analyzing aqueous liquid for corrosion inhibitor concentration in accordance with some implementations of the present disclosure.

The oil field system 200 can include a crude oil pipeline 204. The LC subsystem 100 can be coupled to a crude oil pipeline 204 at a crude oil pipeline tap 202a.

The oil field system 200 can include a gas oil separation unit (GOSP) 201. In general, a GOSP 201 is a continuous separation process used to refine crude oil. The GOSP 201 includes a high pressure production trap (HPPT) 206, a low pressure production trap (LPPT) 208, a low pressure degassing tank (LPDT) 212, a dehydrator unit 210, desalting units 216, and a water/oil separation plant (WOSEP) 214, as well as other components, such as a stabilizer column, centrifugal pumps, heat exchangers, and reboilers. The GOSP 201 can include a crude oil outlet 218.

The LC subsystem 100 can be coupled to various positions of the GOSP 201. For example, the LC subsystem 100 can be coupled to an outlet 202b of the HPPT 206, an outlet 202c of the degassing tank 212, an outlet 202d of the WOSEP 214. Other locations can also be coupled to test corrosion inhibitor concentrations in aqueous liquid through the GOSP 201.

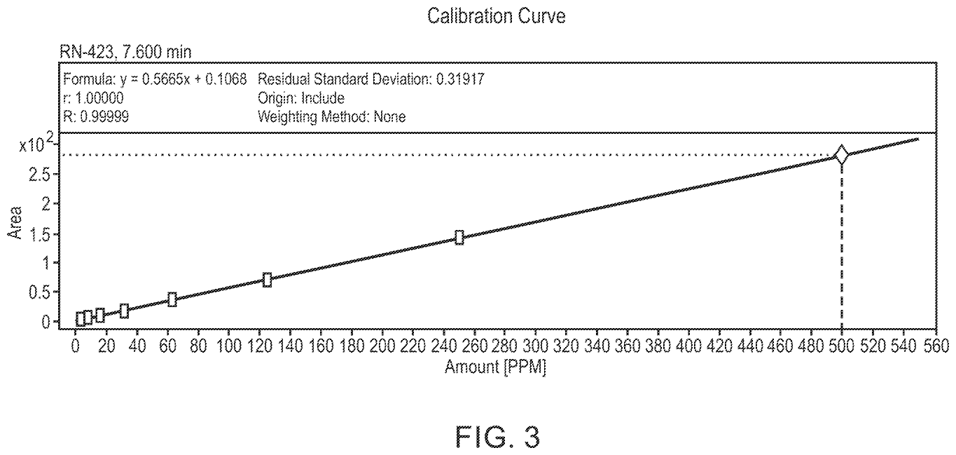

FIG. 3 is a graphical representation of an example calibration curve 300 for establishing a relative concentration of the mobile phase in accordance with some implementations of the present disclosure. Parameters of the calibration curve for the concentrations between 1 mg/L to 500 mg/L is also established. The correlation coefficient obtained was 0.999 and the limit of detection was 1.0. FIG. 4 is an example chromatogram 400 showing retention time of corrosion inhibitors samples using the corrosion inhibitor detector in accordance with some implementations of the present disclosure. Results were the establishment and validation of the analytical conditions of the quantitation of corrosion inhibitor in oil field fluid samples. Also, sensitivity and linearity of the method have established.

Table 3 is an example of concentrations for various calibration levels.

| Calibration | | | | | Concentration |
|---|---|---|---|---|---|
| level | Name | RN [min] | RF | Area | ppm |
| 1 | Corr. Inh | 7.64 | 0.483 | 1.89 | 4.0 |
| 2 | Corr. Inh | 7.62 | 0.595 | 4.812 | 8.0 |
| 3 | Corr. Inh | 7.63 | 0.582 | 9.026 | 15.5 |
| 4 | Corr. Inh | 7.63 | 0.566 | 17.556 | 31.0 |
| 5 | Corr. Inh | 7.62 | 0.57 | 35.689 | 62.0 |
| 6 | Corr. Inh | 7.62 | 0.568 | 70.957 | 125.0 |
| 7 | Corr. Inh | 7.62 | 0.569 | 142.232 | 250.0 |
| 8 | Corr. Inh | 7.61 | 0.567 | 283.062 | 500.0 |

Three trials were run and the measured concentration of corrosion inhibitor was compared to a standardized measurement in produced water. Table 4 depicts the results of the three trials for various concentrations of corrosion inhibitor as compared to the standardized measurement (in parts-per-million (ppm)) with the standard deviation and relative standard deviation.

TABLE 4
Example Results of Three Trials.

| STD ppm | Trial-1 | Trial-2 | Trial-3 | Std. Dev. | RSD (%) |
|---|---|---|---|---|---|
| 1 | 1.2 | 1.2 | 0.9 | 0.06 | 4.95 |
| 5 | 5.2 | 4.9 | 4.8 | 0.21 | 4.19 |
| 10 | 10.3 | 10 | 10.3 | 0.17 | 1.70 |
| 50 | 50.5 | 48.9 | 50.1 | 0.83 | 1.67 |
| 100 | 102 | 97.9 | 100.2 | 2.06 | 2.05 |
| 250 | 252 | 254 | 248 | 3.06 | 1.22 |
| 500 | 497 | 498 | 505 | 4.36 | 0.87 |

An average of the three trials was calculated and compared to the standardized measurement to further illustrate the accuracy of the residual corrosion measurements. Table 5 depicts the absolute error between the standardized measurement and determined average measurement (in ppm):

TABLE 5
Absolute Error between standardized measurement and average
measurement

| STD ppm | Result ppm | Error |
|---|---|---|
| 1 | 1.03 | 3 |
| 5 | 5.12 | 2.4 |
| 10 | 10.3 | 3 |
| 50 | 50.5 | 1 |
| 100 | 102 | 2 |
| 250 | 252 | 0.8 |
| 500 | 497 | −0.6 |

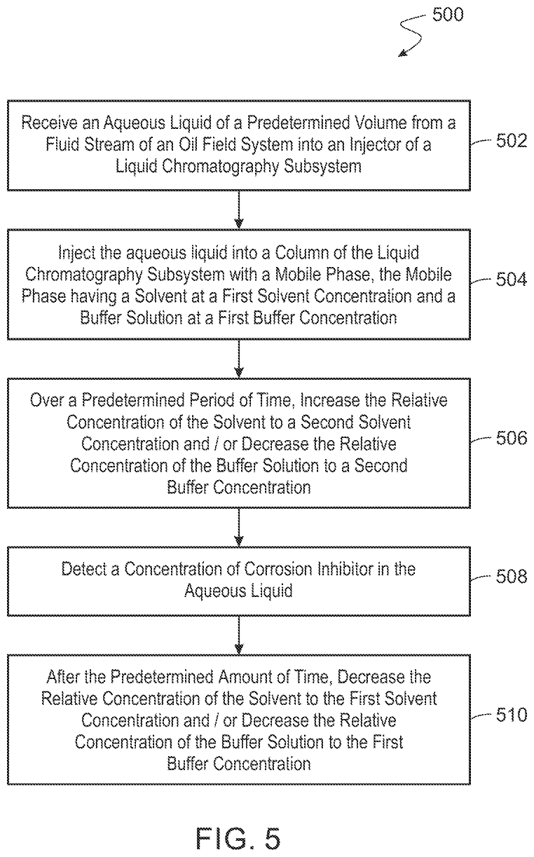

FIG. 5 is a process flow diagram for detecting a concentration of corrosion inhibitor in an aqueous liquid from a fluid stream of an oil field system in accordance with some implementations of the present disclosure. FIG. 5 is a flowchart of an example of a method 500 for detecting concentrations of corrosion inhibitor in an aqueous liquid from a fluid stream of an oil field system according to some implementations of the present disclosure. For clarity of presentation, the description that follows generally describes method 500 in the context of the other figures in this description. However, it will be understood that method 500 can be performed, for example, by any suitable system, environment, software, and hardware, or a combination of systems, environments, software, and hardware, as appropriate. In some implementations, various steps of method 500 can be run in parallel, in combination, in loops, or in any order.

At 502, an LC subsystem can receive an aqueous liquid of a predetermined volume from a fluid stream of an oil field system into an injector of a liquid chromatography subsystem. The LC subsystem can receive the aqueous liquid from a fluid stream at one of various locations of the oil field system. The LC subsystem can receive the aqueous liquid into an injector of the LC subsystem. The volume of aqueous liquid can be controlled by a metering pump of an autosampler coupled to the oil field system.

From 502, method 500 proceeds to 504.

At 504, the volume of aqueous liquid can be injected into a column of the LC subsystem with the mobile phase. The mobile phase including a concentration of solvent at a first solvent concentration and a buffer solution at a first buffer solution concentration. For example, the first solvent concentration can include 25% solvent (or between 24-26%); and the first buffer solution concentration can include 75% buffer solution (or between 74-76%). The solvent can include acetonitrile. The buffer solution can include ammonium acetate.

From 504, method 500 proceeds to 506.

At 506, over a first predetermined period of time, the relative concentration of the solvent can be increased to a second solvent concentration. In some implementations, the relative concentration of the buffer solution can be decreased to a second buffer concentration. For example, the second solvent concentration can include 85% solvent (or between 84-86%); and the second buffer solution concentration can include 15% buffer solution (or between 14-16%). The first predetermined amount of time can include 25 minutes or between 24-26 minutes.

From 506, method 500 proceeds to 508.

At 508, during and after the first predetermined amount of time, the detector can detect a concentration of corrosion inhibitor in the aqueous liquid exiting the column.

From 508, method 500 proceeds to 510.

At 510, over a second predetermined period of time, the relative concentration of the solvent can be decreased to the first solvent concentration. In some implementations, the relative concentration of the buffer solution can be increased to the first buffer concentration. The second predetermined period of time can be 4 to 6 minutes, or 5 minutes.

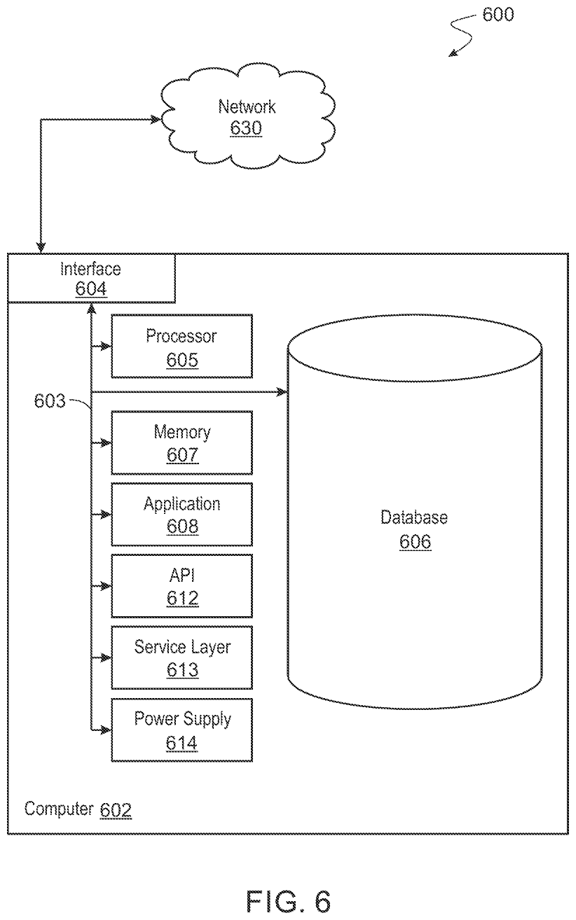

FIG. 6 is a block diagram illustrating an example computer system used to provide computational functionalities associated with described algorithms, methods, functions, processes, flows, and procedures as described in the present disclosure, according to some implementations of the present disclosure. FIG. 6 is a block diagram of an example computer system 600 used to provide computational functionalities associated with described algorithms, methods, functions, processes, flows, and procedures described in the present disclosure, according to some implementations of the present disclosure. The illustrated computer 602 is intended to encompass any computing device such as a server, a desktop computer, a laptop/notebook computer, a wireless data port, a smart phone, a personal data assistant (PDA), a tablet computing device, or one or more processors within these devices, including physical instances, virtual instances, or both. The computer 602 can include input devices such as keypads, keyboards, and touch screens that can accept user information. Also, the computer 602 can include output devices that can convey information associated with the operation of the computer 602. The information can include digital data, visual data, audio information, or a combination of information. The information can be presented in a graphical user interface (UI) (or GUI).

The computer 602 can serve in a role as a client, a network component, a server, a database, a persistency, or components of a computer system for performing the subject matter described in the present disclosure. The illustrated computer 602 is communicably coupled with a network 630. In some implementations, one or more components of the computer 602 can be configured to operate within different environments, including cloud-computing-based environments, local environments, global environments, and combinations of environments.

At a top level, the computer 602 is an electronic computing device operable to receive, transmit, process, store, and manage data and information associated with the described subject matter. According to some implementations, the computer 602 can also include, or be communicably coupled with, an application server, an email server, a web server, a caching server, a streaming data server, or a combination of servers.

The computer 602 can receive requests over network 630 from a client application (for example, executing on another computer 602). The computer 602 can respond to the received requests by processing the received requests using software applications. Requests can also be sent to the computer 602 from internal users (for example, from a command console), external (or third) parties, automated applications, entities, individuals, systems, and computers.

Each of the components of the computer 602 can communicate using a system bus 603. In some implementations, any or all of the components of the computer 602, including hardware or software components, can interface with each other or the interface 604 (or a combination of both) over the system bus 603. Interfaces can use an application programming interface (API) 612, a service layer 613, or a combination of the API 612 and service layer 613. The API 612 can include specifications for routines, data structures, and object classes. The API 612 can be either computer-language independent or dependent. The API 612 can refer to a complete interface, a single function, or a set of APIs.

The service layer 613 can provide software services to the computer 602 and other components (whether illustrated or not) that are communicably coupled to the computer 602. The functionality of the computer 602 can be accessible for all service consumers using this service layer. Software services, such as those provided by the service layer 613, can provide reusable, defined functionalities through a defined interface. For example, the interface can be software written in JAVA, C++, or a language providing data in extensible markup language (XML) format. While illustrated as an integrated component of the computer 602, in alternative implementations, the API 612 or the service layer 613 can be stand-alone components in relation to other components of the computer 602 and other components communicably coupled to the computer 602. Moreover, any or all parts of the API 612 or the service layer 613 can be implemented as child or sub-modules of another software module, enterprise application, or hardware module without departing from the scope of the present disclosure.

The computer 602 includes an interface 604. Although illustrated as a single interface 604 in FIG. 6, two or more interfaces 604 can be used according to particular needs, desires, or particular implementations of the computer 602 and the described functionality. The interface 604 can be used by the computer 602 for communicating with other systems that are connected to the network 630 (whether illustrated or not) in a distributed environment. Generally, the interface 604 can include, or be implemented using, logic encoded in software or hardware (or a combination of software and hardware) operable to communicate with the network 630. More specifically, the interface 604 can include software supporting one or more communication protocols associated with communications. As such, the network 630 or the interface's hardware can be operable to communicate physical signals within and outside of the illustrated computer 602.

The computer 602 includes a processor 605. Although illustrated as a single processor 605 in FIG. 6, two or more processors 605 can be used according to particular needs, desires, or particular implementations of the computer 602 and the described functionality. Generally, the processor 605 can execute instructions and can manipulate data to perform the operations of the computer 602, including operations using algorithms, methods, functions, processes, flows, and procedures as described in the present disclosure.

The computer 602 also includes a database 606 that can hold data for the computer 602 and other components connected to the network 630 (whether illustrated or not). For example, database 606 can be an in-memory, conventional, or a database storing data consistent with the present disclosure. In some implementations, database 606 can be a combination of two or more different database types (for example, hybrid in-memory and conventional databases) according to particular needs, desires, or particular implementations of the computer 602 and the described functionality. Although illustrated as a single database 606 in FIG. 6, two or more databases (of the same, different, or combination of types) can be used according to particular needs, desires, or particular implementations of the computer 602 and the described functionality. While database 606 is illustrated as an internal component of the computer 602, in alternative implementations, database 606 can be external to the computer 602.

The computer 602 also includes a memory 607 that can hold data for the computer 602 or a combination of components connected to the network 630 (whether illustrated or not). Memory 607 can store any data consistent with the present disclosure. In some implementations, memory 607 can be a combination of two or more different types of memory (for example, a combination of semiconductor and magnetic storage) according to particular needs, desires, or particular implementations of the computer 602 and the described functionality. Although illustrated as a single memory 607 in FIG. 6, two or more memories 607 (of the same, different, or combination of types) can be used according to particular needs, desires, or particular implementations of the computer 602 and the described functionality. While memory 607 is illustrated as an internal component of the computer 602, in alternative implementations, memory 607 can be external to the computer 602.

The application 608 can be an algorithmic software engine providing functionality according to particular needs, desires, or particular implementations of the computer 602 and the described functionality. For example, application 608 can serve as one or more components, modules, or applications. Further, although illustrated as a single application 608, the application 608 can be implemented as multiple applications 608 on the computer 602. In addition, although illustrated as internal to the computer 602, in alternative implementations, the application 608 can be external to the computer 602.

The computer 602 can also include a power supply 614. The power supply 614 can include a rechargeable or non-rechargeable battery that can be configured to be either user- or non-user-replaceable. In some implementations, the power supply 614 can include power-conversion and management circuits, including recharging, standby, and power management functionalities. In some implementations, the power-supply 614 can include a power plug to allow the computer 602 to be plugged into a wall socket or a power source to, for example, power the computer 602 or recharge a rechargeable battery.

There can be any number of computers 602 associated with, or external to, a computer system containing computer 602, with each computer 602 communicating over network 630. Further, the terms “client,” “user,” and other appropriate terminology can be used interchangeably, as appropriate, without departing from the scope of the present disclosure. Moreover, the present disclosure contemplates that many users can use one computer 602 and one user can use multiple computers 602.

Described implementations of the subject matter can include one or more features, alone or in combination.

For example, in a first implementation, a computer-implemented method includes the following.

Example 1 a method for determining the concentration of a corrosion inhibitor, the method includes receiving an aqueous liquid of a predetermined volume from a fluid stream of an oil field system into an injector of a liquid chromatography system, the aqueous liquid including a corrosion inhibitor, the liquid chromatography system in fluid communication with the fluid stream of the oil field system, the liquid chromatography system including a stationary phase and a mobile phase, the mobile phase including a solvent at a first solvent concentration range and a buffer at first buffer concentration range; injecting, by the injector, the aqueous liquid of the predetermined volume and the mobile phase into a column of the liquid chromatography system; increasing, by a controller controlling a pump system, the first solvent concentration range to a second solvent concentration range over a first predetermined amount of time, and decreasing, by the controller controlling a pump system, the first buffer concentration range to a second buffer concentration range over the first predetermined amount of time; determining, by a detector of the liquid chromatography system, a concentration of corrosion inhibitor in the aqueous liquid flowing out of the column; and decreasing, by the controller controlling the pump system, the second solvent concentration range to the first solvent concentration range over a second predetermined amount of time, and increasing, by the controller controlling the pump system, the second buffer concentration range to the first buffer concentration range over the second predetermined amount of time.

Example 2 may include the subject matter of example 1, wherein the first solvent concentration range includes a concentration of solvent in the mobile phase ranging from 24% to 26% and the first buffer concentration range includes a concentration of buffer solution in the mobile phase ranging from 74% to 76%.

Example 3 may include the subject matter of any of examples 1-2, wherein the second solvent concentration range includes a concentration of solvent in the mobile phase ranging from 84% to 86% and the second buffer concentration range includes a concentration of buffer solution in the mobile phase ranging from 14% to 16%.

Example 4 may include the subject matter of any of examples 1-3, wherein the first predetermined amount of time is between 24 and 26 minutes.

Example 4 may include the subject matter of any of examples 1-4, wherein the injector injects the aqueous liquid of the predetermined volume and the mobile phase into a column of the liquid chromatography system at a flow rate of approximately 0.8 milliliters per minute (mL/min).

Example 6 may include the subject matter of any of examples 1-5, wherein the predetermined volume includes approximately 1 microliter of aqueous liquid.

Example 7 may include the subject matter of any of examples 1-6, wherein the detector operates at 280 nanometers (nm) wavelength and a reference wavelength of 600 nanometers.

Example 8 may include the subject matter of any of examples 1-7, wherein the autosampler controls a metering pump to sample the predetermined volume of the aqueous liquid from the fluid stream.

Example 9 may include the subject matter of example 8, wherein the metering pump is coupled to a sample reservoir in fluid communication with the fluid stream, the sample reservoir to receive aqueous liquid from the fluid stream through a reservoir inlet and return aqueous fluid to the fluid stream through a reservoir return outlet.

Example 10 may include the subject matter of any of examples 1-9, wherein the fluid stream of the oil field system includes one or more of a fluid stream flowing out of water-oil-separation tank, a fluid stream flowing out of a water degassing tank, a fluid flowing through a crude oil inlet to a gas-oil separation plant (GOSP), or a fluid flowing through a crude oil transfer pipeline.

Example 11 may include the subject matter of any of examples 1-10, wherein the second predetermined amount of time is between 4 and 6 minutes.

Example 12 may include the subject matter of any of examples 1-11, wherein the solvent includes acetonitrile.

Example 13 may include the subject matter of any of examples 1-12, wherein the buffer includes ammonium acetate.

Example 14 may include the subject matter of any of examples 1-13, wherein the corrosion inhibitor includes one or more of amides, imidazoline, salts of nitrogenous molecules, nitrogen quaternaries, polyoxyalkylated amines, amides and imidazolines, nitrogen heterocyclic.

Example 15 may include the subject matter of any of examples 1-14, wherein the stationary phase is non-polar.

Example 16 is a detection system for determining a concentration of corrosion inhibitor in an aqueous liquid from a fluid steam of an oil field system, the detection system includes a liquid chromatography (LC) subsystem that includes an LC column including a non-polar stationary phase, an injector to inject a mobile phase and aqueous liquid into the LC column, a controller to control a time rate of change of a relative concentration of solvent and buffer solution in the mobile phase, a detector to detect a concentration of corrosion inhibitor in the aqueous liquid flowing from the column during operation of the LC subsystem, and an autosampler to receive aqueous liquid from a fluid stream of the oil field system and to deliver the aqueous liquid to the liquid chromatography subsystem. The detection system also includes a fluid inlet coupler to couple an inlet of the autosampler to an outlet of an asset of the oil field system, the asset containing the fluid stream.

Example 17 may include the subject matter of example 16, further including a metering pump downstream of the autosampler to control the volume of aqueous liquid delivered to the liquid chromatography subsystem.

Example 18 may include the subject matter of any of examples 16-17, further including a pump system controlled by the controller to pump the solvent and the buffer into the injector, the controller to control the pump system to increase a relative concentration of the solvent from 25% to 85% over 25 minutes and to decrease a relative concentration of the buffer solution from 75% to 15% over the 25 minutes.

Example 19 may include the subject matter of any of examples 16-18, wherein the fluid inlet coupler is configured to couple to the asset of the oil field system at one or more of an outlet of a water-oil-separation tank, an outlet of a water degassing tank, an outlet of a gas-oil separation plant (GOSP), a crude oil transfer pipeline, or an outlet of a high-pressure production trap.

Example 20 may include the subject matter of any of examples 16-19, wherein the liquid chromatography subsystem includes a reverse phase liquid chromatography (RPLC) subsystem.

Implementations of the subject matter and the functional operations described in this specification can be implemented in digital electronic circuitry, in tangibly embodied computer software or firmware, in computer hardware, including the structures disclosed in this specification and their structural equivalents, or in combinations of one or more of them. Software implementations of the described subject matter can be implemented as one or more computer programs. Each computer program can include one or more modules of computer program instructions encoded on a tangible, non-transitory, computer-readable computer-storage medium for execution by, or to control the operation of, data processing apparatus. Alternatively, or additionally, the program instructions can be encoded in/on an artificially generated propagated signal. For example, the signal can be a machine-generated electrical, optical, or electromagnetic signal that is generated to encode information for transmission to a suitable receiver apparatus for execution by a data processing apparatus. The computer-storage medium can be a machine-readable storage device, a machine-readable storage substrate, a random or serial access memory device, or a combination of computer-storage mediums.

The terms “data processing apparatus,” “computer,” and “electronic computer device” (or equivalent as understood by one of ordinary skill in the art) refer to data processing hardware. For example, a data processing apparatus can encompass all kinds of apparatuses, devices, and machines for processing data, including by way of example, a programmable processor, a computer, or multiple processors or computers. The apparatus can also include special purpose logic circuitry including, for example, a central processing unit (CPU), a field-programmable gate array (FPGA), or an application-specific integrated circuit (ASIC). In some implementations, the data processing apparatus or special purpose logic circuitry (or a combination of the data processing apparatus or special purpose logic circuitry) can be hardware- or software-based (or a combination of both hardware- and software-based). The apparatus can optionally include code that creates an execution environment for computer programs, for example, code that constitutes processor firmware, a protocol stack, a database management system, an operating system, or a combination of execution environments. The present disclosure contemplates the use of data processing apparatuses with or without conventional operating systems, such as LINUX, UNIX, WINDOWS, MAC OS, ANDROID, or IOS.

A computer program, which can also be referred to or described as a program, software, a software application, a module, a software module, a script, or code, can be written in any form of programming language. Programming languages can include, for example, compiled languages, interpreted languages, declarative languages, or procedural languages. Programs can be deployed in any form, including as stand-alone programs, modules, components, subroutines, or units for use in a computing environment. A computer program can, but need not, correspond to a file in a file system. A program can be stored in a portion of a file that holds other programs or data, for example, one or more scripts stored in a markup language document, in a single file dedicated to the program in question, or in multiple coordinated files storing one or more modules, sub-programs, or portions of code. A computer program can be deployed for execution on one computer or on multiple computers that are located, for example, at one site or distributed across multiple sites that are interconnected by a communication network. While portions of the programs illustrated in the various figures may be shown as individual modules that implement the various features and functionality through various objects, methods, or processes, the programs can instead include a number of sub-modules, third-party services, components, and libraries. Conversely, the features and functionality of various components can be combined into single components as appropriate. Thresholds used to make computational determinations can be statically, dynamically, or both statically and dynamically determined.

The methods, processes, or logic flows described in this specification can be performed by one or more programmable computers executing one or more computer programs to perform functions by operating on input data and generating output. The methods, processes, or logic flows can also be performed by, and apparatus can also be implemented as, special purpose logic circuitry, for example, a CPU, an FPGA, or an ASIC.

Computers suitable for the execution of a computer program can be based on one or more of general and special purpose microprocessors and other kinds of CPUs. The elements of a computer are a CPU for performing or executing instructions and one or more memory devices for storing instructions and data. Generally, a CPU can receive instructions and data from (and write data to) a memory.

Graphics processing units (GPUs) can also be used in combination with CPUs. The GPUs can provide specialized processing that occurs in parallel to processing performed by CPUs. The specialized processing can include artificial intelligence (AI) applications and processing, for example. GPUs can be used in GPU clusters or in multi-GPU computing.

A computer can include, or be operatively coupled to, one or more mass storage devices for storing data. In some implementations, a computer can receive data from, and transfer data to, the mass storage devices including, for example, magnetic, magneto-optical disks, or optical disks. Moreover, a computer can be embedded in another device, for example, a mobile telephone, a personal digital assistant (PDA), a mobile audio or video player, a game console, a global positioning system (GPS) receiver, or a portable storage device such as a universal serial bus (USB) flash drive.

Computer-readable media (transitory or non-transitory, as appropriate) suitable for storing computer program instructions and data can include all forms of permanent/non-permanent and volatile/non-volatile memory, media, and memory devices. Computer-readable media can include, for example, semiconductor memory devices such as random access memory (RAM), read-only memory (ROM), phase change memory (PRAM), static random access memory (SRAM), dynamic random access memory (DRAM), erasable programmable read-only memory (EPROM), electrically erasable programmable read-only memory (EEPROM), and flash memory devices. Computer-readable media can also include, for example, magnetic devices such as tape, cartridges, cassettes, and internal/removable disks. Computer-readable media can also include magneto-optical disks and optical memory devices and technologies including, for example, digital video disc (DVD), CD-ROM, DVD+/−R, DVD-RAM, DVD-ROM, HD-DVD, and BLU-RAY. The memory can store various objects or data, including caches, classes, frameworks, applications, modules, backup data, jobs, web pages, web page templates, data structures, database tables, repositories, and dynamic information. Types of objects and data stored in memory can include parameters, variables, algorithms, instructions, rules, constraints, and references. Additionally, the memory can include logs, policies, security or access data, and reporting files. The processor and the memory can be supplemented by, or incorporated into, special purpose logic circuitry.

Implementations of the subject matter described in the present disclosure can be implemented on a computer having a display device for providing interaction with a user, including displaying information to (and receiving input from) the user. Types of display devices can include, for example, a cathode ray tube (CRT), a liquid crystal display (LCD), a light-emitting diode (LED), and a plasma monitor. Display devices can include a keyboard and pointing devices including, for example, a mouse, a trackball, or a trackpad. User input can also be provided to the computer through the use of a touchscreen, such as a tablet computer surface with pressure sensitivity or a multi-touch screen using capacitive or electric sensing. Other kinds of devices can be used to provide for interaction with a user, including to receive user feedback including, for example, sensory feedback including visual feedback, auditory feedback, or tactile feedback. Input from the user can be received in the form of acoustic, speech, or tactile input. In addition, a computer can interact with a user by sending documents to, and receiving documents from, a device that the user uses. For example, the computer can send web pages to a web browser on a user's client device in response to requests received from the web browser.

The term “graphical user interface,” or “GUI,” can be used in the singular or the plural to describe one or more graphical user interfaces and each of the displays of a particular graphical user interface. Therefore, a GUI can represent any graphical user interface, including, but not limited to, a web browser, a touch-screen, or a command line interface (CLI) that processes information and efficiently presents the information results to the user. In general, a GUI can include a plurality of user interface (UI) elements, some or all associated with a web browser, such as interactive fields, pull-down lists, and buttons. These and other UI elements can be related to or represent the functions of the web browser.

Implementations of the subject matter described in this specification can be implemented in a computing system that includes a back-end component, for example, as a data server, or that includes a middleware component, for example, an application server. Moreover, the computing system can include a front-end component, for example, a client computer having one or both of a graphical user interface or a Web browser through which a user can interact with the computer. The components of the system can be interconnected by any form or medium of wireline or wireless digital data communication (or a combination of data communication) in a communication network. Examples of communication networks include a local area network (LAN), a radio access network (RAN), a metropolitan area network (MAN), a wide area network (WAN), Worldwide Interoperability for Microwave Access (WIMAX), a wireless local area network (WLAN) (for example, using 802.11 a/b/g/n or 802.20 or a combination of protocols), all or a portion of the Internet, or any other communication system or systems at one or more locations (or a combination of communication networks). The network can communicate with, for example, Internet Protocol (IP) packets, frame relay frames, asynchronous transfer mode (ATM) cells, voice, video, data, or a combination of communication types between network addresses.

The computing system can include clients and servers. A client and server can generally be remote from each other and can typically interact through a communication network. The relationship of client and server can arise by virtue of computer programs running on the respective computers and having a client-server relationship.

Cluster file systems can be any file system type accessible from multiple servers for read and update. Locking or consistency tracking may not be necessary since the locking of exchange file system can be done at application layer. Furthermore, Unicode data files can be different from non-Unicode data files.

While this specification contains many specific implementation details, these should not be construed as limitations on the scope of what may be claimed, but rather as descriptions of features that may be specific to particular implementations. Certain features that are described in this specification in the context of separate implementations can also be implemented, in combination, in a single implementation. Conversely, various features that are described in the context of a single implementation can also be implemented in multiple implementations, separately, or in any suitable sub-combination. Moreover, although previously described features may be described as acting in certain combinations and even initially claimed as such, one or more features from a claimed combination can, in some cases, be excised from the combination, and the claimed combination may be directed to a sub-combination or variation of a sub-combination.

Particular implementations of the subject matter have been described. Other implementations, alterations, and permutations of the described implementations are within the scope of the following claims as will be apparent to those skilled in the art. While operations are depicted in the drawings or claims in a particular order, this should not be understood as requiring that such operations be performed in the particular order shown or in sequential order, or that all illustrated operations be performed (some operations may be considered optional), to achieve desirable results. In certain circumstances, multitasking or parallel processing (or a combination of multitasking and parallel processing) may be advantageous and performed as deemed appropriate.

Moreover, the separation or integration of various system modules and components in the previously described implementations should not be understood as requiring such separation or integration in all implementations. It should be understood that the described program components and systems can generally be integrated together in a single software product or packaged into multiple software products.

Accordingly, the previously described example implementations do not define or constrain the present disclosure. Other changes, substitutions, and alterations are also possible without departing from the spirit and scope of the present disclosure.

Furthermore, any claimed implementation is considered to be applicable to at least a computer-implemented method; a non-transitory, computer-readable medium storing computer-readable instructions to perform the computer-implemented method; and a computer system including a computer memory interoperably coupled with a hardware processor configured to perform the computer-implemented method or the instructions stored on the non-transitory, computer-readable medium.

What is claimed is:

1. A method for determining the concentration of a corrosion inhibitor, comprising:
receiving an aqueous liquid of a predetermined volume from a fluid stream of an oil field system into an injector of a liquid chromatography system, the aqueous liquid comprising a corrosion inhibitor, the liquid chromatography system in fluid communication with the fluid stream of the oil field system, the liquid chromatography system comprising a stationary phase and a mobile phase, the mobile phase comprising a solvent at a first solvent concentration range and a buffer at first buffer concentration range;
injecting, by the injector, the aqueous liquid of the predetermined volume and the mobile phase into a column of the liquid chromatography system;
increasing, by a controller controlling a pump system, the first solvent concentration range to a second solvent concentration range over a first predetermined amount of time, and decreasing, by the controller controlling a pump system, the first buffer concentration range to a second buffer concentration range over the first predetermined amount of time;
determining, by a detector of the liquid chromatography system, a concentration of corrosion inhibitor in the aqueous liquid flowing out of the column; and
decreasing, by the controller controlling the pump system, the second solvent concentration range to the first solvent concentration range over a second predetermined amount of time, and increasing, by the controller controlling the pump system, the second buffer concentration range to the first buffer concentration range over the second predetermined amount of time.

2. The method of claim 1, wherein the first solvent concentration range comprises a concentration of solvent in the mobile phase ranging from 24% to 26% and the first buffer concentration range comprises a concentration of buffer solution in the mobile phase ranging from 74% to 76%.

3. The method of claim 1, wherein the second solvent concentration range comprises a concentration of solvent in the mobile phase ranging from 84% to 86% and the second buffer concentration range comprises a concentration of buffer solution in the mobile phase ranging from 14% to 16%.

4. The method of claim 1, where in the first predetermined amount of time is between 24 and 26 minutes.

5. The method of claim 1, wherein the injector injects the aqueous liquid of the predetermined volume and the mobile phase into a column of the liquid chromatography system at a flow rate of approximately 0.8 milliliters per minute (mL/min).

6. The method of claim 1, wherein the predetermined volume comprises approximately 1 microliter of aqueous liquid.

7. The method of claim 1, wherein the detector operates at 280 nanometers (nm) wavelength and a reference wavelength of 600 nanometers.

8. The method of claim 1, wherein the autosampler controls a metering pump to sample the predetermined volume of the aqueous liquid from the fluid stream.

9. The method of claim 8, wherein the metering pump is coupled to a sample reservoir in fluid communication with the fluid stream, the sample reservoir to receive aqueous liquid from the fluid stream through a reservoir inlet and return aqueous fluid to the fluid stream through a reservoir return outlet.

10. The method of claim 1, wherein the fluid stream of the oil field system comprises one or more of a fluid stream flowing out of water-oil-separation tank, a fluid stream flowing out of a water degassing tank, a fluid flowing through a crude oil inlet to a gas-oil separation plant (GOSP), or a fluid flowing through a crude oil transfer pipeline.

11. The method of claim 1, wherein the second predetermined amount of time is between 4 and 6 minutes.

12. The method of claim 1, wherein the solvent comprises acetonitrile.

13. The method of claim 1, wherein the buffer comprises ammonium acetate.

14. The method of claim 1, wherein the corrosion inhibitor comprises one or more of amides, imidazoline, salts of nitrogenous molecules, nitrogen quaternaries, polyoxyalkylated amines, amides and imidazolines, nitrogen heterocyclic.

15. The method of claim 1, wherein the stationary phase is non-polar.

16. A detection system for determining a concentration of corrosion inhibitor in an aqueous liquid from a fluid steam of an oil field system, the detection system comprising:
a liquid chromatography (LC) subsystem comprising:
an LC column comprising a non-polar stationary phase,
an injector to inject a mobile phase and aqueous liquid into the LC column,
a controller to control a time rate of change of a relative concentration of solvent and buffer solution in the mobile phase,
a detector to detect a concentration of corrosion inhibitor in the aqueous liquid flowing from the column during operation of the LC subsystem, and
an autosampler to receive aqueous liquid from a fluid stream of the oil field system and to deliver the aqueous liquid to the liquid chromatography subsystem; and
a fluid inlet coupler to couple an inlet of the autosampler to an outlet of an asset of the oil field system, the asset containing the fluid stream.

17. The detection system of claim 16, further comprising a metering pump downstream of the autosampler to control the volume of aqueous liquid delivered to the liquid chromatography subsystem.

18. The detection system of claim 16, further comprising a pump system controlled by the controller to pump the solvent and the buffer into the injector, the controller to control the pump system to increase a relative concentration of the solvent from 25% to 85% over 25 minutes and to decrease a relative concentration of the buffer solution from 75% to 15% over the 25 minutes.

19. The detection system of claim 16, wherein the fluid inlet coupler is configured to couple to the asset of the oil field system at one or more of an outlet of a water-oil-separation tank, an outlet of a water degassing tank, an outlet of a gas-oil separation plant (GOSP), a crude oil transfer pipeline, or an outlet of a high-pressure production trap.

20. The detection system of claim 16, wherein the liquid chromatography subsystem comprises a reverse phase liquid chromatography (RPLC) subsystem.
